(19) United States
(12) Patent Application Publication
(10) Pub. No.: US 2015/0289842 A1
(43) Pub. Date: Oct. 15, 2015

(54) ULTRASONIC GENERATING DEVICE

(71) Applicant: NATIONAL TAIWAN UNIVERSITY, TAIPEI (TW)

(72) Inventors: Chih-Yu CHAO, TAIPEI (TW); Horng-Huei LIOU, TAIPEI (TW); Wei-Ting CHEN, TAIPEI (TW); Bo-Lun MA, TAIPEI (TW)

(21) Appl. No.: 14/534,191

(22) Filed: Nov. 6, 2014

(30) Foreign Application Priority Data
Apr. 11, 2014 (TW) 103113449

(51) Int. Cl. A61B 8/00; A61B 8/14

(52) U.S. Cl. CPC A61B 8/4494; A61B 8/145

(57) ABSTRACT

An ultrasonic generating device, which can generate an ultrasound with complex waveform, includes a first waveform generating element, which emits a first waveform, and a second waveform generating element. The second waveform generating element periodically adjusts the first waveform with at least one frequency higher than the first waveform to form a second waveform in the first waveform. The two waveforms are superposed to form the ultrasound with complex waveform. By means of the penetrating ability of the first waveform, the second waveform can be carried into the object region with the complex waveform, therefore have biological medical effects.

### RELATED APPLICATIONS

This application claims priority to Taiwanese Application Serial Number 103113449, filed Apr. 11, 2014, which is herein incorporated by reference.

### BACKGROUND

1. Field of Invention

The present invention relates to an ultrasonic generating device. More particularly, the present invention relates to a complex waveform ultrasonic generating device.

2. Description of Related Art

Ultrasound is an ultrasonic wave with frequency over 20,000 Hz, which cannot be heard by human ear. The applications of the ultrasound include ultrasonic cleaning, ultrasonic measuring, ultrasonic welding and ultrasonic diagnosis in many aspects. In medical treatments, ultrasound with different frequencies is chosen for different purposes. The commonly used frequency of the ultrasound is about 1 MHz to 10 MHz, and the major application of the ultrasound includes diagnosis, thermal therapy, shock wave lithotripsy, liposuction and so on.

In terms of the ultrasonic medical treatment, high-frequency ultrasound can promote neural stem cell renewal in human body. However, the high-frequency ultrasound has no penetrating ability and prone to decay in the human body, an invasive treatment is needed to transmit the high-frequency ultrasound into the human body. In the application of the ultrasonic diagnosis, the ultrasound with lower frequency is used for transmitting the ultrasound into the body to obtain the image of a specific area. Because the high-frequency ultrasound cannot be used inside the body, the resolution of the ultrasonic image cannot be enhanced.

### SUMMARY

Therefore, in various embodiments of the present disclosure, an ultrasonic generating device is provided, which can generate an ultrasound with complex waveform. Thus the generated ultrasound may penetrate as the low frequency ultrasound and have high resolution of the high-frequency ultrasound. The ultrasonic generating device can apply in ultrasonic imaging and send high-frequency ultrasounds into an object for medical application.

One aspect of the present disclosure is an ultrasonic generating device for generating an ultrasound with complex waveform including: a first waveform generating element emitting a first waveform; and a second waveform generating element periodically adjusting the first waveform with at least one frequency higher than that of the first waveform to form a second waveform in the first waveform, and the two waveforms are superposed to form the ultrasound with complex waveform, wherein a frequency of the second waveform is in a range from about 15 MHz to about 250 THz, and a frequency of the first waveform is in a range from about 15 MHz to about 150 kHz.

According to various embodiments of the present disclosure, the ultrasound with complex waveform has biological medical effects.

According to various embodiments of the present disclosure, the first and the second waveforms are selected from a group consisting of the following waveforms: a square wave, a sinusoidal wave, a triangular wave, a pulse wave and combinations thereof.

According to various embodiments of the present disclosure, a amplitude, frequency, period, a duration time of the first and second waveforms can be adjusted, and a time for turn on/off a first waveform generating element and a second waveform generating element can also be controlled.

According to various embodiments of the present disclosure, the first waveform generating element is an ultrasonic generating material.

According to various embodiments of the present disclosure, the second waveform generating element is an oscillating material, and the oscillating material is distributed into the ultrasonic generating material or coupled to the ultrasonic generating material.

According to various embodiments of the present disclosure, the ultrasonic generating material is a first piezoelectric material.

According to various embodiments of the present disclosure, the first piezoelectric material is selected from a group consisting of quartz, tourmaline, Rochelle salt, tantalate, niobate, titanate and combinations thereof.

According to various embodiments of the present disclosure, the first piezoelectric material is selected from a group consisting of lithium tantalate, lithium niobate, tantalum niobate, strontium niobate, strontium barium niobate barium lead titanate, lead titanate, barium titanate, lead zirconate titanate and combinations thereof.

According to various embodiments of the present disclosure, the first piezoelectric material is a piezoelectric thin-film material or a polymer piezoelectric film.

According to various embodiments of the present disclosure, the piezoelectric thin-film material is selected from a group consisting of aluminum nitride, zinc oxide, polyvinylidene fluoride (PVDF) and combinations thereof.

According to various embodiments of the present disclosure, the oscillating material is a phononic crystal, a semiconductor material, or a second piezoelectric material, wherein a frequency of a waveform generated by the second piezoelectric material is larger than that of a waveform generated by the first piezoelectric material.

According to various embodiments of the present disclosure, the second piezoelectric material is a piezoelectric thin-film material or a polymer piezoelectric film.

According to various embodiments of the present disclosure, the piezoelectric thin-film material is selected from a group consisting of aluminum nitride, zinc oxide, polyvinylidene fluoride (PVDF) and combinations thereof.

According to various embodiments of the present disclosure, the second piezoelectric material is selected from a group consisting of quartz, tourmaline, Rochelle salt, tantalate, niobate, titanate and combinations thereof.

According to various embodiments of the present disclosure, the second piezoelectric material is selected from a group consisting of lithium tantalate, lithium niobate, tantalum niobate, strontium niobate, strontium barium niobate barium lead titanate, lead titanate, barium titanate, lead zirconate titanate and combinations thereof.

According to various embodiments of the present disclosure, the semiconductor material is selected from a group consisting of gallium nitride, Indium gallium nitride, silicon carbide and combinations thereof.

According to various embodiments of the present disclosure, the first waveform generating element is a first ultrasonic transmitter.

According to various embodiments of the present disclosure, the second waveform generating element is a periodical waveform regulator, and the periodical waveform regulator connects with the first ultrasonic transmitter.

According to various embodiments of the present disclosure, the periodical waveform regulator is selected from a group consisting of a second ultrasonic transmitter, a filter, a function generator, a high-frequency switch, a chopper and combinations thereof.

According to various embodiments of the present disclosure, the second ultrasonic transmitter is a pulsed ultrasonic transmitter.

According to various embodiments of the present disclosure, the ultrasonic generating device further including a phase-oppositing ultrasonic transmitter emitting a phase-oppositing ultrasound which is out-of-phase relative to that of the first waveform, and a pathway of the phase-oppositing ultrasound overlaps partially or totally with that of the ultrasound with complex waveform.

An aspect of the present disclosure is an ultrasonic imaging device including: an ultrasonic array transmitter including a plurality of ultrasonic generating devices, the ultrasonic generating device including: a first waveform generating element emitting a first waveform, and a second waveform generating element periodically adjusting the first waveform with at least one frequency higher than that of the first waveform to form a second waveform in the first waveform, and the two waveforms are superposed to form an ultrasound with complex waveform, wherein a frequency of the second waveform is in a range from about 15 MHz to about 250 THz, and a frequency of the first waveform is in a range from about 15 MHz to about 150 kHz; and an ultrasonic array receiver including a plurality of ultrasonic receiving devices to receive the ultrasonic with complex waveform reflecting from an area to be imaged.

According to various embodiments of the present disclosure, a frequency and an amplitude of the first waveform and second waveform in the ultrasound with complex waveform can be adjusted.

According to various embodiments of the present disclosure, the ultrasonic imaging device is used in an ultrasonic operation using ultrasound to assist orientation and a 3D or 4D real-time imaging.

Another aspect of the present disclosure is a method for generating an ultrasound with complex waveform, including following operations. A first waveform is formed. A second waveform is formed in the first waveform, and the two waveforms are superposed to form the ultrasound with complex waveform. A frequency of the second waveform is in a range from about 15 MHz to about 250 THz, and a frequency of the first waveform is in a range from about 15 MHz to about 150 kHz.

According to various embodiments of the present disclosure, the operation of forming a first waveform includes using an ultrasonic transmitter or an ultrasonic generating material to emit the first waveform.

According to various embodiments of the present disclosure, the operation of forming a second waveform in the first waveform includes using a femtoseond laser to excite a second waveform generating element to emit the second waveform.

According to various embodiments of the present disclosure, the operation of forming a second waveform in the first waveform includes using a second waveform generating element to adjust the first waveform periodically.

According to various embodiments of the present disclosure, the operation of using a second waveform generating element to adjust the first waveform periodically includes using the second waveform generating element to periodically cancel, turn off, or regulate the first waveform.

According to various embodiments of the present disclosure, the operation of using the second waveform generating element to periodically regulate the first waveform includes doping, casting, blending, epitaxy growing, or coupling the second waveform generating element in the first waveform generating element. The first waveform generating element is an ultrasonic generating material, and the second waveform generating element is an oscillating material.

According to various embodiments of the present disclosure, the first waveform generating element is an ultrasonic transmitter and the second waveform generating element is a high-frequency switch.

According to various embodiments of the present disclosure, the operation of using the second waveform generating element to periodically cancel the first waveform includes emitting a pulsed ultrasound to cancel part of the first waveform to form the second waveform in the first waveform. The second waveform generating element is selected from a group consisting of a chopper, a filter, a function generator, and combinations thereof.

According to various embodiments of the present disclosure, the method is applied in an ultrasonic imaging and medical application having biological effect.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a schematic diagram of an ultrasound with complex waveform according to various embodiments of the present disclosure;

FIG. 2 is a schematic diagram of an ultrasonic generating device according to various embodiments of the present disclosure;

FIG. 3 is a schematic diagram of an ultrasonic generating device according to various embodiments of the present disclosure;

FIG. 4 is a schematic diagram of an ultrasonic generating device according to various embodiments of the present disclosure;

FIG. 5 is a schematic diagram of an ultrasound with complex waveform according to various embodiments of the present disclosure;

FIG. 6 is a schematic diagram of an ultrasound with complex waveform according to various embodiments of the present disclosure;

FIG. 7 is a schematic diagram of an ultrasound with complex waveform according to various embodiments of the present disclosure;

FIG. 8 is a schematic diagram of an ultrasonic generating device according to various embodiments of the present disclosure;

FIG. 9 is a schematic diagram of an ultrasonic generating device according to various embodiments of the present disclosure;

FIG. 10 is a schematic diagram of an ultrasonic generating device according to various embodiments of the present disclosure;

FIG. 11 is a schematic diagram of an ultrasonic generating device according to various embodiments of the present disclosure;

FIG. 12 is a schematic diagram of an ultrasonic generating device according to various embodiments of the present disclosure;

FIG. 13 is a schematic diagram of an ultrasound waveform according to various embodiments of the present disclosure;

FIG. 14 is a schematic diagram of an ultrasound generating device according to various embodiments of the present disclosure;

FIG. 15 is a schematic diagram of an ultrasonic imaging device according to various embodiments of the present disclosure; and

FIG. 16 is a schematic diagram of an ultrasonic imaging device according to various embodiments of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

As used herein, the terms “comprising,” “including,” “having,” “involving,” and the like are to be understood to be open-ended, i.e., to mean including but not limited to.

The singular forms “a,” “an” and “the” used herein include plural referents unless the context clearly dictates otherwise. Therefore, reference to, for example, a dielectric layer includes embodiments having two or more such dielectric layers, unless the context clearly indicates otherwise.

Referring to FIG. 1, FIG. 1 is a schematic diagram of an ultrasound with complex waveform according to various embodiments of the present disclosure. An ultrasound with complex waveform 100 includes a first waveform 102 having an amplitude A2 and period t3, and a second waveform 104 having an amplitude A1 and period t1, t2. The first waveform is a low-frequency waveform, and a frequency of the first waveform is in a range from about 15 MHz to about 150 kHz. The first waveform can penetrate into human body and reach a specific organ or tissue. The second waveform is a high-frequency waveform, and a frequency of the second waveform is in a range from about 15 MHz to about 250 THz. In various embodiments of the present disclosure, an ultrasonic generating device which can generate an ultrasonic with complex waveform is provided, and the amplitude A2 and period t3 of the first waveform and the amplitude A1 and period t1, t2 of the second waveform can be adjusted by the device. In various embodiments of the present disclosure, the period t1, t2 can be independently adjusted. For example, the second waveform is generated by a second waveform generating element, wherein the period t1 is the time the second waveform generating element turned on, and the period t2 is the time the second waveform generating element turned off. By switching the second waveform generating element, a second waveform can be formed in the first waveform, and t1, t2 can be adjusted independently. In various embodiments of the present disclosure, the second waveform generating element can use a frequency higher than that of the first waveform to periodically adjust the first waveform, in which the period of the frequency is t1+t2, and forms the second waveform in the first waveform. In various embodiments of the present disclosure, the first waveform is emitted from a first waveform generating element, and the period of the first waveform t3 can be controlled by a switch of the first waveform generating element. In various embodiments of the present disclosure, the duration time of the first and second waveforms can also be controlled by the switch of the first waveform generating element and the second waveform generating element. Because a high-frequency ultrasound has no penetrating ability, which can not penetrate into human body and reach an object, an ultrasonic generating device for generating an ultrasound with complex waveform is provided. The high-frequency ultrasound can be sent into a specific object by the ultrasound with complex waveform, and the application of the device is a non-invasive method. In various embodiments of the present disclosure, the ultrasound with complex waveform has biological medical effects. The biological medical effects include a biological effect or a medical effect generated by the high-frequency ultrasounds oscillating in a cell or tissue. In various embodiments of the present disclosure, the ultrasound with complex waveform may be a square wave or a sinusoidal wave. In various embodiments of the present disclosure, the ultrasound with complex waveform may be a triangular wave, a pulse wave, or a composite waveform formed by superpositing the square wave, the sinusoidal wave, the triangular wave, and the pulse wave after design or calculation.

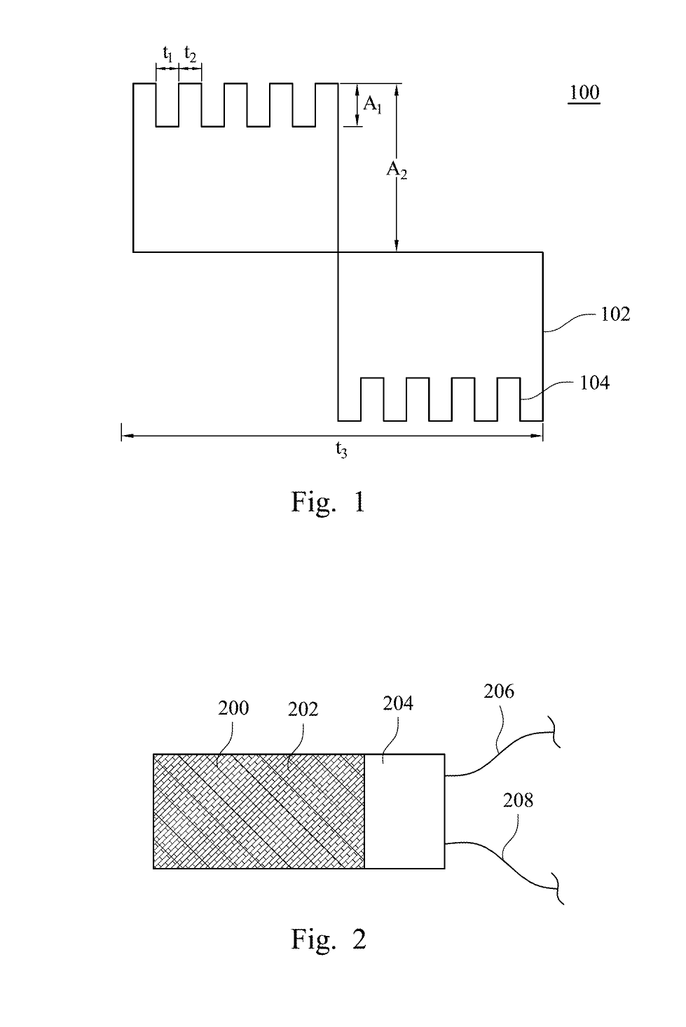

Referring to FIG. 2, FIG. 2 is a schematic diagram of an ultrasonic generating device according to various embodiments of the present disclosure. The ultrasonic generating device includes a first waveform generating element and a second waveform generating element. The first waveform generating element is used to emit a first waveform, and the second waveform generating element is used to periodically adjust the first waveform with at least one frequency higher than that of the first waveform to form a second waveform in the first waveform, and the two waveforms are superposed to form the ultrasound with complex waveform. In various embodiments of the present disclosure, the ultrasonic generating device is an ultrasonic transmitter, and the first waveform generating element is an ultrasonic generating material 200. In various embodiments of the present disclosure, the ultrasonic generating material is a first piezoelectric material, which can transfer electric energy to material oscillation so as to emit an ultrasound. In various embodiments of the present disclosure, the first piezoelectric material is a piezoelectric thin-film material or a polymer piezoelectric film. The piezoelectric thin-film material includes aluminum nitride and zinc oxide; the polymer piezoelectric film includes for example polyvinylidene fluoride (PVDF) film. The first piezoelectric material may be piezoelectric material including quartz, tourmaline, Rochelle salt, tantalate (such as lithium tantalate), niobate (such as lithium niobate, tantalum niobate, strontium barium niobate and strontium niobate), or titanate (such as barium lead titanate, lead titanate, barium titanate, lead zirconate titanate). In various embodiments of the present disclosure, the second waveform generating element is an oscillating material 202. The oscillating material 202 may be distributed into the ultrasonic generating material 200 by doping, casting, or blending, also may use epitaxy growing device, such as chemical vapor deposition (CVD), molecular beam epitaxy (MBE), liquid phase epitaxy (LPE) and solid phase epitaxy (SPE) to grow the oscillating material 202 in the piezoelectric material. And the CVD may subdivide into many kinds of CVD depending on the technical features, such as different operating pressure, different gas phase features, and plasma enhanced or not, including ultra-high vacuum chemical vapor deposition (UHVCVD), aerosol-assisted chemical vapor deposition (AACVD), plasma-enhanced chemical vapor deposition, metal organic chemical vapor deposition (MOCVD) and so on. The oscillating material 202 may generate an oscillation having frequency higher than that of the ultrasonic generating material 200 when it is electrified. Therefore the oscillating material 202 generates the second waveform in the first waveform generated by the ultrasonic generating material 200. In various embodiments of the present disclosure, the ultrasonic generating device includes the ultrasonic generating material 200; the oscillating material 202 distributed into the ultrasonic generating material 200; a support 204 connected with the ultrasonic generating material 200 for handheld; and a wire 206 connecting to and electrically oscillating the ultrasonic generating material 200 and the oscillating material 202 for emitting an ultrasound with complex waveform. In various embodiments of the present disclosure, the ultrasonic generating device further includes another wire 208 to provide another voltage, making the oscillating material 202 oscillate with different frequency. The frequency and the amplitude of the second waveform may be controlled by adjusting the voltage, the kind of the oscillating material 202, or the growing method of the oscillating material 202. In various embodiments of the present disclosure, the ultrasonic generating device includes a laser, which is used to excite the oscillating material 202 for emitting high-frequency ultrasound. In various embodiments of the present disclosure, the oscillating material 202 may be a phononic crystal, a semiconductor material, or a second piezoelectric material, wherein the frequency of the waveform generated by the second piezoelectric material is higher than that of the first piezoelectric material. The semiconductor material includes gallium nitride, Indium gallium nitride, silicon carbide and so on. In various embodiments of the present disclosure, the second piezoelectric material is a piezoelectric thin-film material or a polymer piezoelectric film. The piezoelectric thin-film material includes aluminum nitride and zinc oxide; the polymer piezoelectric film includes for example polyvinylidene fluoride (PVDF) film. The second piezoelectric material may be the piezoelectric material including quartz, tourmaline, Rochelle salt, tantalate (such as lithium tantalate), niobate (such as lithium niobate, tantalum niobate, strontium barium niobate and strontium niobate), or titanate (such as barium lead titanate, lead titanate, barium titanate, lead zirconate titanate). The oscillating material 202 may be distributed into the first piezoelectric material by doping, casting, blending, or above-mentioned epitaxy growing method, and generates a in-phase or a out-of-phase oscillation relative to that of the first piezoelectric material, to generate the second waveform in the first waveform and form an ultrasound with complex waveform.

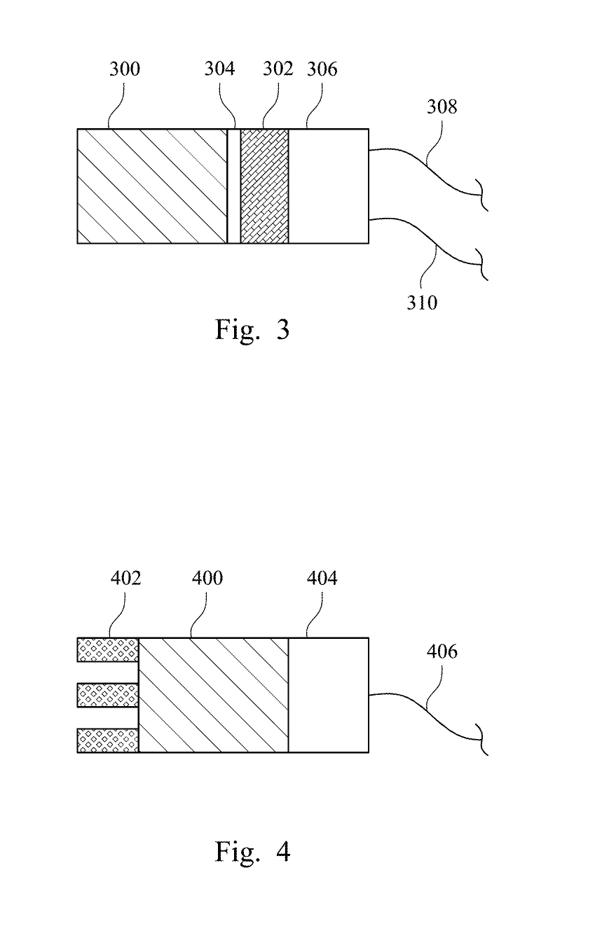

Referring to FIG. 3, FIG. 3 is a schematic diagram of an ultrasonic generating device according to various embodiments of the present disclosure. In various embodiments of the present disclosure, a first waveform generating element is an ultrasonic generating material 300. In various embodiments of the present disclosure, the ultrasonic generating material 300 is a first piezoelectric material, which can transfer electric energy to a material oscillation so as to emit an ultrasound. In various embodiments of the present disclosure, the first piezoelectric material is a piezoelectric thin-film material or a polymer piezoelectric film. The piezoelectric thin-film material includes aluminum nitride and zinc oxide; the polymer piezoelectric film includes for example polyvinylidene fluoride (PVDF) film. The first piezoelectric material may be a piezoelectric material including quartz, tourmaline, Rochelle salt, tantalate (such as lithium tantalate), niobate (such as lithium niobate, tantalum niobate, strontium barium niobate and strontium niobate), or titanate (such as barium lead titanate, lead titanate, barium titanate, lead zirconate titanate). In various embodiments of the present disclosure, the second waveform generating element is an oscillating material 302. The oscillating material 302 may be a phononic crystal, a semiconductor material, or a second piezoelectric material, wherein the frequency of the waveform generated by the second piezoelectric material is higher than that of the first piezoelectric material. The semiconductor material includes gallium nitride, Indium gallium nitride, silicon carbide and so on. In various embodiments of the present disclosure, the second piezoelectric material is a piezoelectric thin-film material or a polymer piezoelectric film. The piezoelectric thin-film material includes aluminum nitride and zinc oxide; the polymer piezoelectric film includes for example polyvinylidene fluoride (PVDF) film. The second piezoelectric material may be piezoelectric material including quartz, tourmaline, Rochelle salt, tantalate (such as lithium tantalate), niobate (such as lithium niobate, tantalum niobate, strontium barium niobate and strontium niobate), or titanate (such as barium lead titanate, lead titanate, barium titanate, lead zirconate titanate). The ultrasonic generating material 300 and the oscillating material 302 are coupled by a coupling agent 304. The oscillating material 302 connects with a support 306, which is for handhold. Support 306 further connects to a wire 308 to connect electricity and makes the ultrasonic generating material 300 and the oscillating material 302 oscillate to emit an ultrasound with complex waveform. In various embodiments of the present disclosure, the ultrasonic generating device further includes another wire 310, which provides a different voltage and let the oscillating material 302 oscillate with different frequency. In various embodiments of the present disclosure, the ultrasonic generating device includes a laser, which is used to excite the oscillating material 302 for emitting high-frequency ultrasound. The high-frequency ultrasound generated by the oscillating material 302 can superpose with the first waveform generated by the ultrasonic generating material to form the second waveform in the first waveform and forms the ultrasound with complex waveform.

Referring to FIG. 4, FIG. 4 is a schematic diagram of an ultrasonic generating device according to various embodiments of the present disclosure. In various embodiments of the present disclosure, the first waveform generating element is an ultrasonic generating material 400. In various embodiments of the present disclosure, the ultrasonic generating material is the first piezoelectric material, which can transfer electric energy to material oscillation so as to emit an ultrasound. The first piezoelectric material may be the above-mentioned piezoelectric material such as tantalum niobate etc. In various embodiments of the present disclosure, the oscillating material 402 is a phononic crystal with multilayer structure. The phononic crystal may be a one dimensional, two dimensional, or three dimensional phononic crystal, and has a periodic lattice structure. In the field of solid-state electronics, a semiconductor which is confined in all three spatial dimensions is called quantum dots. Quantum dots have electron behavior similar to an artificial atom. Therefore, arranging the phononic crystal in a specific lattice orientation, designing the dimension, symmetry, arrangement, distribution, and concentration of the phononic crystal, then connecting the phononic crystal with part of the ultrasonic generating material 400 makes part of ultrasound passing through the phononic crystal having a different high-frequency oscillation, and forms the second waveform in the first waveform. In various embodiments of the present disclosure, ultrasonic generating material 400 connects with the support 404, which is for handheld. Support 404 further connects with the wire 406. When the wire connects to electricity, the ultrasonic generating material 400 and the oscillating material 402 may generate coupled oscillation and emit the ultrasound with complex waveform. The ultrasonic generating material 400 may emit the first waveform when is electrified. A second waveform may be formed in the first waveform after part of the first waveform passing through the phononic crystal in front of the ultrasonic generating material 400. The frequency of the second waveform is higher than that of the first waveform, and the second waveform may couple with the first waveform to form the ultrasound with complex waveform.

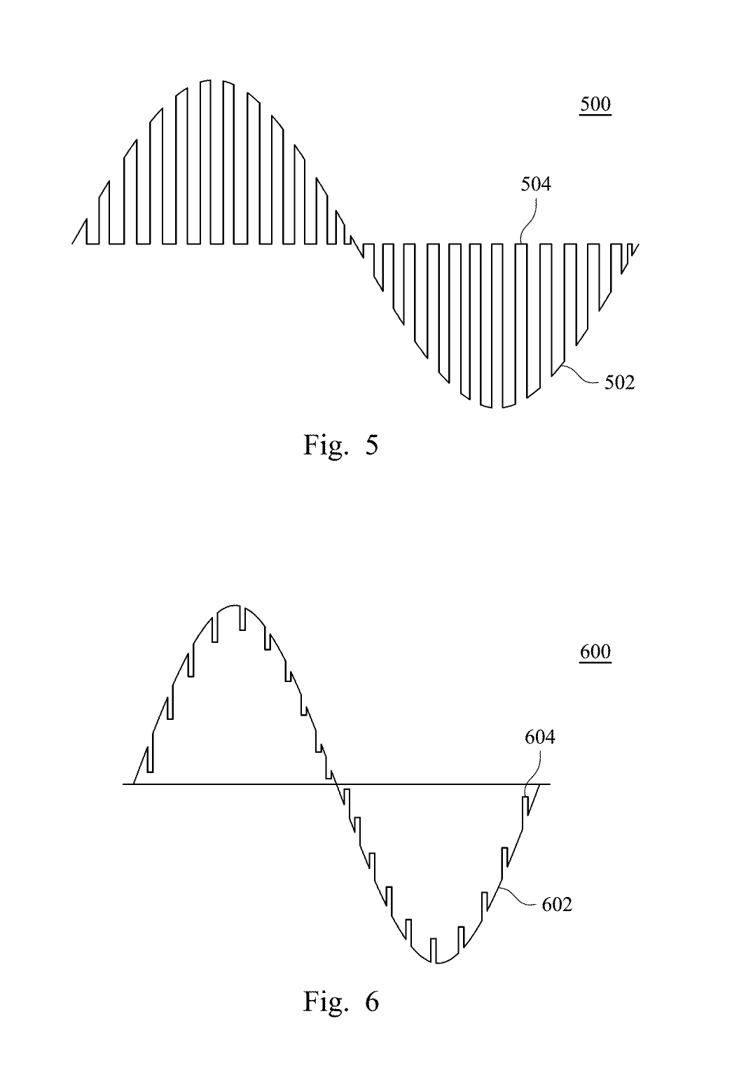

Referring to FIGS. 5-7, FIGS. 5-7 are schematic diagrams of ultrasounds with complex waveforms according to various embodiments shown in FIGS. 8-11 of the present disclosure. In FIGS. 5-7, the second waveform is formed by canceling or eliminating part of the first waveform. FIG. 5 is a schematic diagram of an ultrasound with complex waveform according to various embodiments of the present disclosure. An ultrasound with complex waveform 500 includes a first waveform 502, and a second waveform 504. In various embodiments of the present disclosure, the first waveform is a sinusoidal wave. The first waveform is a low frequency waveform, and the frequency of the first waveform is in a range from about 15 MHz to about 150 kHz. The first waveform can penetrate into human body and will not be completely decayed. The second waveform is a high-frequency waveform, and the frequency of the second waveform is in a range from about 15 MHz to about 250 THz.

Referring to FIG. 6, FIG. 6 is a schematic diagram of an ultrasound with complex waveform according to various embodiments of the present disclosure. An ultrasound with complex waveform 600 includes a first waveform 602, and a second waveform 604. In various embodiments of the present disclosure, the first waveform is a sinusoidal wave. The first waveform is a low-frequency waveform, and the frequency of the first waveform is in a range from about 15 MHz to about 150 kHz. The first waveform can penetrate into human body and will not be completely decayed. The second waveform is a high-frequency waveform, and the frequency of the second waveform is in a range from about 15 MHz to about 250 THz. The difference between the ultrasounds with complex waveforms shown in FIG. 6 and FIG. 5 is that only part of the amplitude of the first waveform is canceled to form the second waveform in the first waveform in FIG. 6.

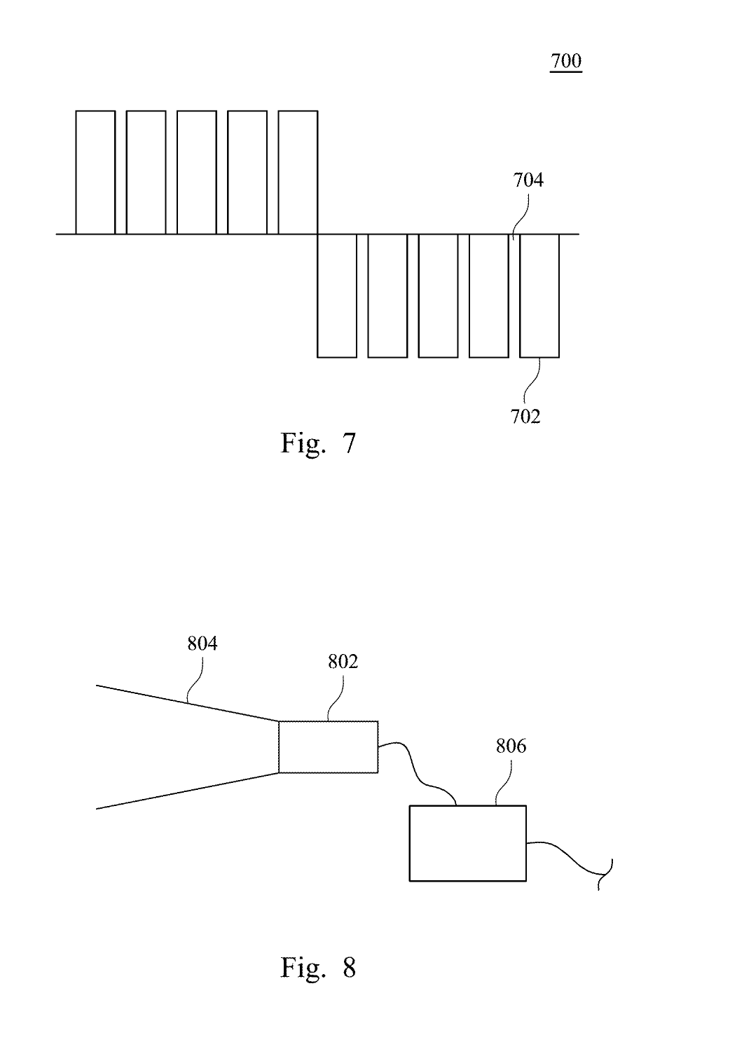

Referring to FIG. 7, FIG. 7 is a schematic diagram of an ultrasound with complex waveform according to various embodiments of the present disclosure. An ultrasound with complex waveform 700 includes a first waveform 702, and a second waveform 704. In various embodiments of the present disclosure, the first waveform is a square wave. The first waveform is a low-frequency waveform, and the frequency of the first waveform is in a range from about 15 MHz to about 150 kHz. The first waveform can penetrate into human body and will not be completely decayed. The second waveform is a high-frequency waveform, and the frequency of the second waveform is in a range from about 15 MHz to about 250 THz. Part of the amplitude in the first waveform has been completely canceled and forms the second waveform.

Referring to FIG. 8, FIG. 8 is a schematic diagram of an ultrasonic generating device according to various embodiments of the present disclosure. The ultrasonic generating device includes a first waveform generating element and a second waveform generating element, which are used to emit a first waveform and adjust the first waveform by canceling part of the first waveform to form a second waveform in the first waveform for forming an ultrasound with complex waveform, separately. Controlling the switch of the second waveform generating element can adjust the frequency and period of the second waveform. In various embodiments of the present disclosure, the first waveform generating element is a first ultrasonic transmitter 802. In various embodiments of the present disclosure, the first ultrasonic transmitter 802 may be chosen to be a first ultrasonic transmitter which can emit a first waveform with desired frequency. In various embodiments of the present disclosure, the second waveform generating element is a periodical waveform regulator. In the illustrated embodiment, the periodical waveform regulator is a filter 806. The filter 806 has functions such as finite impulse response or comb filter to transfer the signal to the spatial domain by Fourier transform, operate with a functional filter, and use an inverse Fourier transform to transfer the signal back to the frequency domain. The filter 806 uses a frequency higher then that of the first waveform to filter the first waveform emitted by the ultrasonic transmitter 802 periodically, and forms the second waveform in the first waveform. The second waveform is superposed with the first waveform to form the ultrasound with complex waveform. Using filter 806 can filter part of the first waveform periodically, to form the second waveform in the first waveform, for forming the ultrasound with complex waveform 804.

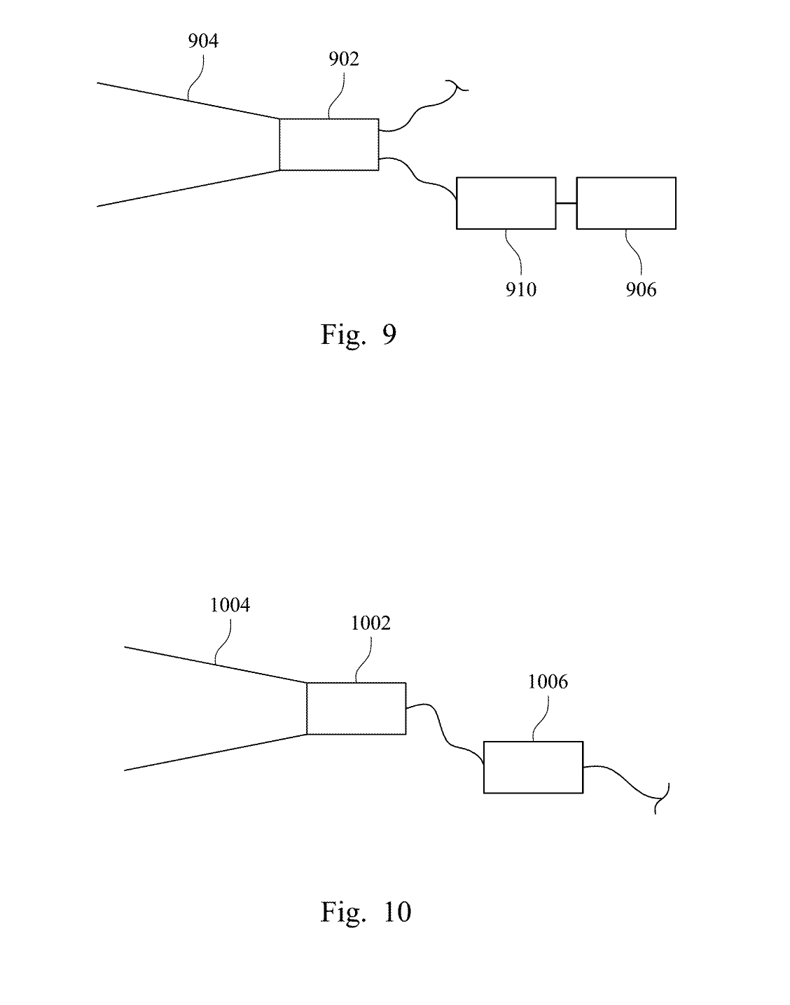

Referring to FIG. 9, FIG. 9 is a schematic diagram of an ultrasonic generating device according to various embodiments of the present disclosure. In some embodiments, the first waveform generating element is a first ultrasonic transmitter 902. In various embodiments of the present disclosure, the second waveform generating element is a periodical waveform regulator. The periodical waveform regulator is a function generator 906. The function generator 906 generates a high frequency signal and sends to the first ultrasonic transmitter 902 for superposing the second waveform in the first waveform which is emitted by the first ultrasonic transmitter 902, to form an ultrasound with complex waveform 904. In various embodiments of the present disclosure, the function generator 906 connects to an amplifier 910 which amplifies a signal and sends the signal to the first ultrasonic transmitter 902. In various embodiments of the present disclosure, the first ultrasonic transmitter 902 may connect to another function generator and another amplifier. Another function generator may generate a signal with frequency lower than that of the function generator 906 to adjust the frequency and amplitude of the first waveform generated by the first ultrasonic transmitter. Therefore, the second waveform of the ultrasound with complex waveform 904 can be adjusted by the function generator 906, and the first waveform of the ultrasound with complex waveform 904 can also be adjusted by another function generator. In various embodiments of the present disclosure, a function generator which can emit two different frequencies can be used. The function generator can connect with two amplifiers which send two different signals to the first ultrasonic transmitter 902 for forming the ultrasound with complex waveform 904.

Referring to FIG. 10, FIG. 10 is a schematic diagram of an ultrasonic generating device according to various embodiments of the present disclosure. A first waveform generating element is a first ultrasonic transmitter 1002. In various embodiments of the present disclosure, the first ultrasonic transmitter 1002 may be chosen to emit a first waveform with desired frequency. In various embodiments of the present disclosure, the second waveform generating element is a periodical waveform regulator. In the illustrated embodiment, the periodical waveform regulator is a high-frequency switch 1006. The high-frequency switch, which can periodically turn on and off in high speed by changing the voltage value, or can control the pulse frequency and the pulse duration time. The high-frequency switch can also be operated with a frequency higher than that of the first waveform, therefore effects the first ultrasonic transmitter 1002, making portions of the generated first waveform being cut off by the high frequency switch, wherein the cut off portion in the first waveform is the second waveform. Therefore using the high-frequency switch makes the ultrasonic generating device emitting an ultrasound with complex waveform 1004. The first waveform is a low-frequency waveform, and the frequency of the first waveform is in a range from about 15 MHz to about 150 kHz. The first waveform can penetrate into human body and will not be completely decayed. The second waveform is a high-frequency waveform, and the frequency of the second waveform is in a range from about 15 MHz to about 250 THz.

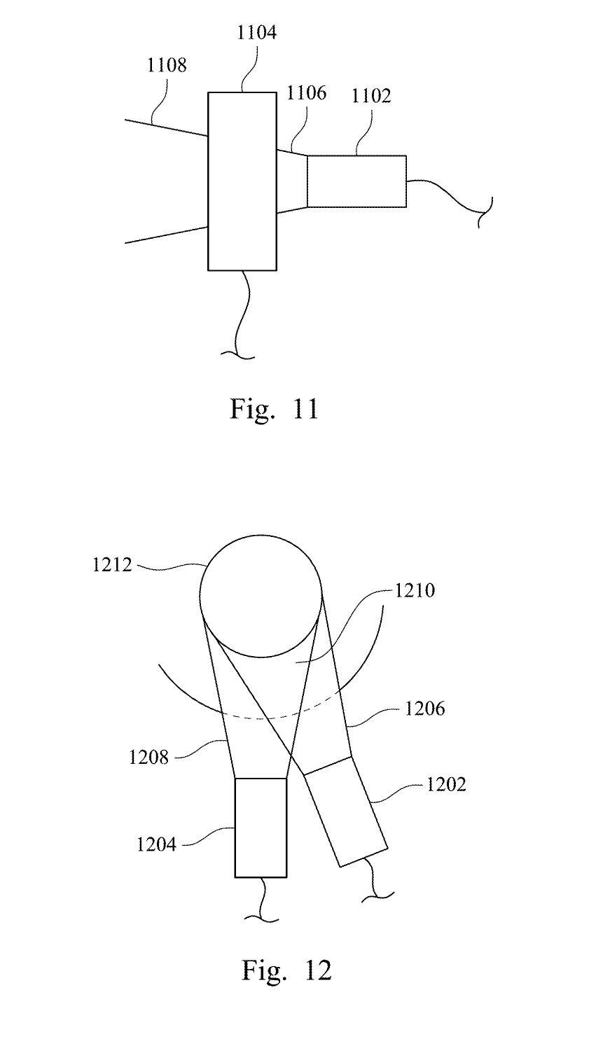

Referring to FIG. 11, FIG. 11 is a schematic diagram of an ultrasonic generating device according to various embodiments of the present disclosure. The first waveform generating element is a first ultrasonic transmitter 1102. In various embodiments of the present disclosure, the first ultrasonic transmitter may be chosen to emit a first waveform with desired frequency. In various embodiments of the present disclosure, the second waveform generating element is a periodical waveform regulator. In the illustrated embodiment, the periodical waveform regulator is a chopper 1104. The chopper 1104 is operated with a frequency higher than that of the first waveform, making the first waveform 1106 emitted by the first ultrasonic transmitter 1002 being chopped with the frequency when it passes through the chopper 1104. The waveform chopped in the first waveform forms the second waveform. The first waveform transfers to an ultrasound with complex waveform 1108 after passing through the chopper. The ultrasound with complex waveform 1108 includes the first waveform with low frequency, and there are more than one high-frequency oscillations (the second waveform) existing in the first waveform. In various embodiments of the present disclosure, the ultrasound with complex waveform 1108 has biological medical effects. The high-frequency ultrasounds with different frequencies have different biological medical effects, such as treating specific cancers, myocardial infarction, or brain diseases.

Referring to FIG. 12, FIG. 12 is a schematic diagram of an ultrasonic generating device according to various embodiments of the present disclosure. The first waveform generating element is a first ultrasonic transmitter 1202. In various embodiments of the present disclosure, the first ultrasonic transmitter 1202 may be chosen to emit a first waveform with desired frequency. In various embodiments of the present disclosure, the second waveform generating element is a periodical waveform regulator. In the illustrated embodiment, the periodical waveform regulator is a second ultrasonic transmitter 1204. The ultrasonic transmitter 1204 is operated with a frequency higher than that of the first waveform. In various embodiments of the present disclosure, the second ultrasonic transmitter 1204 is a pulsed ultrasonic transmitter. In various embodiments of the present disclosure, the first ultrasonic transmitter 1202 emits a first waveform 1206. The second ultrasonic transmitter 1204 emits a pulsed ultrasound 1208 with frequency higher than that of the first waveform. When the pathways of the first waveform 1206 and the pulsed ultrasound 1208 partly or totally overlapped, the pulsed ultrasound 1208 may offset part of the first waveform 1206 due to the wave superposition in the overlapping area. The offseted part of the first waveform is the second waveform, the first and second waveforms form the ultrasound with complex waveform 1210. The ultrasound with complex waveform 1210 can penetrate to the object 1212. The formed waveform of the ultrasound with complex waveform can refer to FIGS. 5-7.

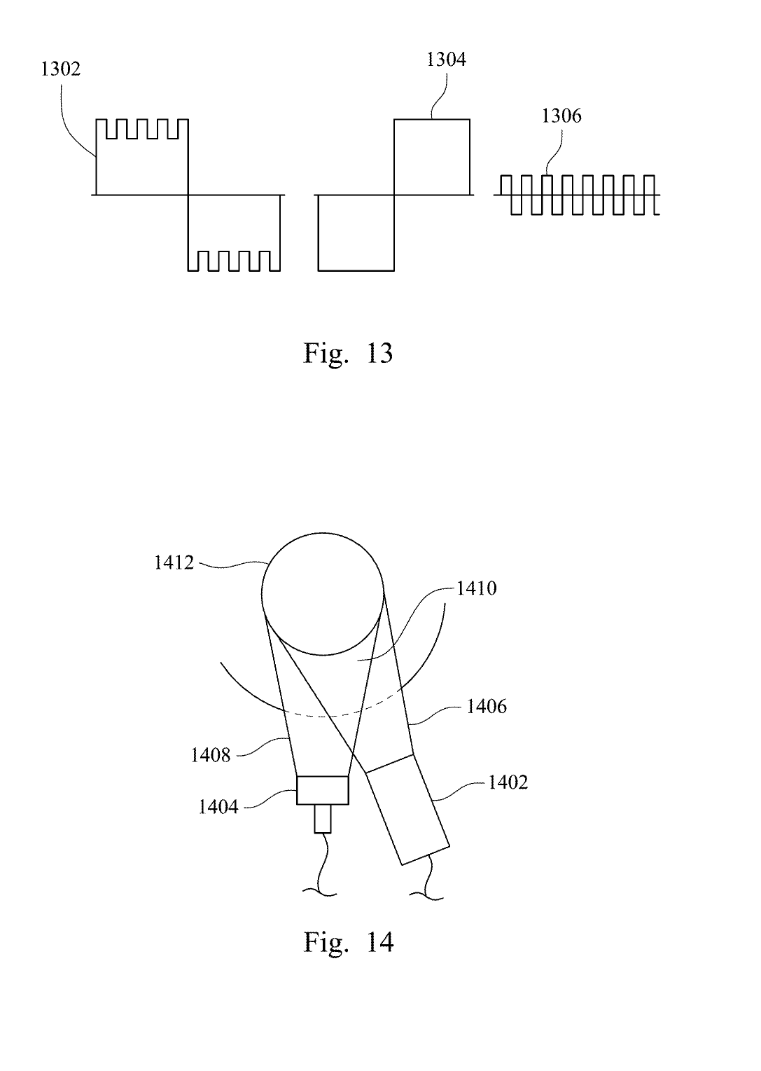

Referring to FIG. 13, FIG. 13 is a schematic diagram of an ultrasonic waveform according to various embodiments of the present disclosure. The figure shows that the low frequency part may be deducted from the complex waveform 1302 after superposing with a phase-oppositing first waveform 1304, which is out-of-phase relative to the low-frequency first waveform, and left a high-frequency second waveform 1306. In various embodiments of the present disclosure, the superposing method can be used to send the high-frequency ultrasonic to the object. The overlapping region of the waveforms 1302 and 1304 will not be affected by the low-frequency waveform, and the method can decrease the side-effects to the tissues in the ultrasonic pathway for using low-frequency ultrasound for a long time, such as the effect of shear force and cavitation effect made by the ultrasound. The first waveform is a low-frequency waveform, and the frequency of the first waveform is in a range from about 15 MHz to about 150 kHz. The first waveform can penetrate into human body and will not decay completely. The second waveform is a high-frequency waveform, and the frequency of the second waveform is in a range from about 15 MHz to about 250 THz.

Referring FIG. 14, FIG. 14 is a schematic diagram of an ultrasonic generating device according to various embodiments of the present disclosure. The figure shows the ultrasonic generating device 1402 may further be used with a phase-oppositing ultrasonic transmitter 1404. The ultrasonic generating device 1402 emits an ultrasound with complex waveform 1406. The ultrasonic transmitter 1404 emits a phase-oppositing first waveform 1408. The pathways of the two waveforms are partially or totally overlapped to form an overlapping area, and the object 1412 is inside the overlapping area. In the overlapping area, the phase-oppositing first waveform 1408 superposes with the ultrasound with complex waveform 1406, and cancels the first waveform in the ultrasound with complex waveform 1406, therefore only the second waveform 1410 is left and sent into the object 1412. The device can send the second waveform, which is a high-frequency ultrasound, into human body, and solves the problem that the penetrating depth of the high-frequency ultrasound is not enough. Besides, combining the phase-oppositing ultrasound with the ultrasonic generating device can cancel the first low-frequency waveform, preventing the low-frequency ultrasound to harm the object. In various embodiments of the present disclosure, the high-frequency ultrasound with different frequencies has different biological medical effects, such as treating specific cancers, myocardial infarction, or brain diseases.

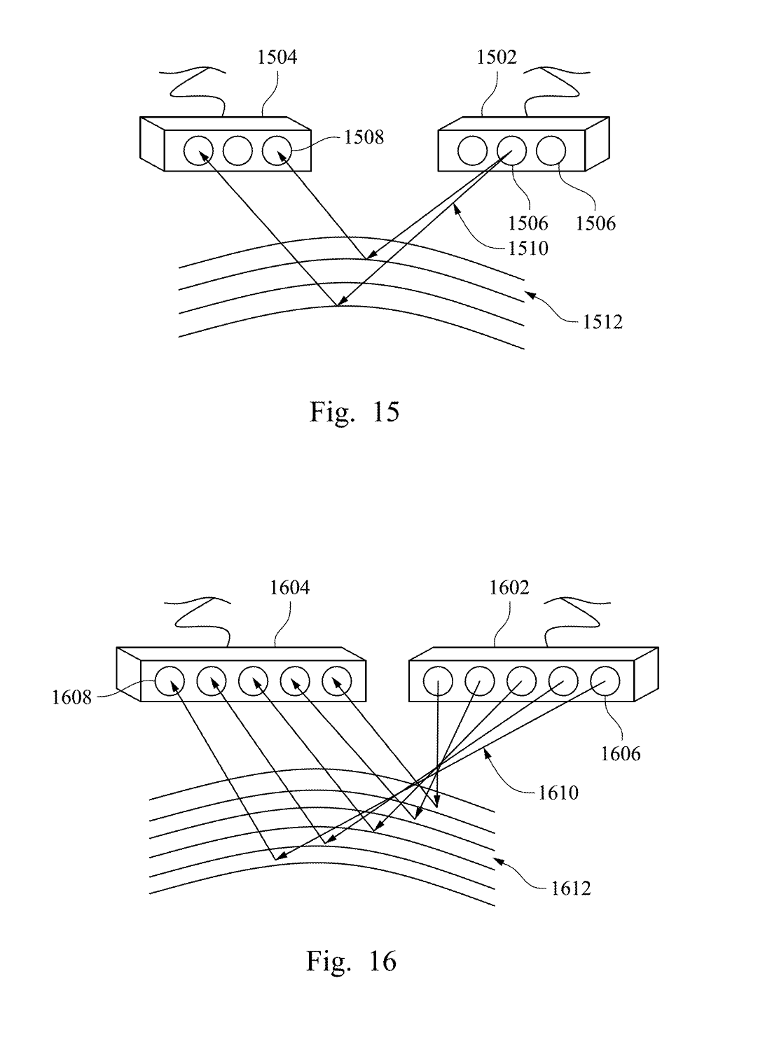

Referring to FIG. 15, FIG. 15 is a schematic diagram of an ultrasonic imaging device according to various embodiments of the present disclosure. An ultrasonic imaging device includes an ultrasonic array transmitter 1502 and an ultrasonic array receiver 1504. The ultrasonic array transmitter includes a plurality of ultrasonic generating devices 1506, and the ultrasonic generating devices 1506 may emit a plurality of ultrasounds with complex waveforms 1510. The ultrasonic generating device 1506 includes a first waveform generating element emitting a first waveform, and a second waveform generating element periodically adjusting the first waveform with at least one frequency higher than that of the first waveform to form a second waveform in the first waveform, and the two waveforms are superposed to form the ultrasound with complex waveform. Wherein the frequency of the second waveform is in a range from about 15 MHz to about 250 THz, and the frequency of the first waveform is in a range from about 15 MHz to about 150 kHz. The details of the ultrasonic generating device may refer to embodiments in FIGS. 2-4 and FIGS. 8-12. The ultrasonic array receiver 1504 includes a plurality of ultrasonic receiving devices 1508 to receive the ultrasound with complex waveform 1510 reflecting form an object 1512 to be imaged. As shown in the figure, the ultrasonic array transmitter 1502 emit ultrasounds with complex waveforms 1510 with the same frequency. The ultrasounds with complex waveforms 1510 reflect at the surface with different depth of the object 1512. And the ultrasonic array receiver 1504 receives the ultrasounds with complex waveforms 1510 reflected by the object 1512 for ultrasonic imaging. The embodiments use the ultrasound with complex waveforms, wherein the first waveform has penetrating ability, which can penetrate into human body and carry the second waveform to the object, then reflect out of human body; and the second waveform is a high-frequency ultrasound, which provides a higher resolution in real time, also enhance the effect of imaging. In various embodiments of the present disclosure, the received ultrasounds with complex waveforms may be treated with numerical analysis and calculating with spatial filtering for high and low frequency to filter the wave with specific frequency, and enhance the resolution of the topical area. In various embodiments of the present disclosure, the ultrasonic generating device 1506 and the ultrasonic receiving device 1508 may integrate in one array. In various embodiments of the present disclosure, every ultrasound with complex waveform emitted by the ultrasonic generating device includes a low-frequency first waveform, and at least one high-frequency second waveform in the first waveform. In operating, the ultrasound with complex waveform which is emitted by the ultrasonic array transmitter is reflected by the object in different depths, and the reflected waveforms are received at different time intervals by the ultrasonic array receiver.

Referring to FIG. 16, FIG. 16 is a schematic diagram of an ultrasonic imaging device according to various embodiments of the present disclosure. As shown in the figure, the ultrasonic imaging device includes an ultrasonic array transmitter 1602 and an ultrasonic array receiver 1604. The ultrasonic array transmitter includes a plurality of ultrasonic generating devices 1606, and the ultrasonic generating devices 1606 may emit a plurality of ultrasounds with complex waveforms 1610. The ultrasounds with complex waveforms 1610 are reflected by the object 1612 and received by the ultrasonic array receiver 1604. The ultrasonic array receiver 1604 includes a plurality of ultrasonic receiving devices 1608. In various embodiments of the present disclosure, the different ultrasonic generating devices 1606 emit ultrasounds with complex waveforms 1610 having different frequencies and amplitudes. The ultrasounds with complex waveforms 1610 with different frequencies may be reflected at the object 1612 with different depths and be received by the ultrasonic array receiver 1604. The ultrasonic imaging device using complex waveforms with different frequencies, waveforms and intensities may be applied in 3D or 4D real-time imaging. The intensity gradient of the ultrasound with complex waveform can be used to detect the tissue in different depths and different reflecting surfaces for the object. Besides, the high-frequency second waveform in the ultrasound with complex waveform can enhance the resolution for the imaging. In various embodiments of the present disclosure, a software calculation can be used with the ultrasonic imaging device, and filters out the unwanted frequency in other regions to enhance the signal strength in the topical area, and enhance the resolution of the ultrasounds. In various embodiments of the present disclosure, the ultrasonic generating device 1606 and the ultrasonic receiving device 1608 can be integrated in one array. In various embodiments of the present disclosure, every ultrasonic generating device emits the ultrasounds with complex waveforms with different intensities, frequencies and amplitudes. Every ultrasounds with complex waveforms emitted by the ultrasonic generating device includes a low-frequency first waveform, and at least one high-frequency second waveform in the first waveform. In operation, the ultrasounds with complex waveforms which are emitted by the ultrasonic array transmitter in the same time are reflected by the object in different depths, and are received at the same time by the ultrasonic array receiver. The received ultrasonic signals can be calculated and analyzed for forming the ultrasonic image. The technique of the present disclosure may apply for 3D or 4D real-time imaging, and can largely enhance the resolution of the 3D or 4D real time imaging.

In various embodiments of the present disclosure, a method for generating an ultrasound with complex waveform is provided, including following operations. A first waveform is formed. And a second waveform is formed in the first waveform, and the two waveforms are superposed to form the ultrasound with complex waveform. Wherein the frequency of the second waveform is in a range from about 15 MHz to about 250 THz, and the frequency of the first waveform is in a range from about 15 MHz to about 150 kHz. The ultrasound with complex waveform may refer to the schematic diagrams for FIGS. 1, 5, 6 and 7. The first waveform and the second waveform may be generated by the same device, or may be generated by a plurality of devices, separately. In various embodiments of the present disclosure, the operation of forming the first waveform includes using an ultrasonic transmitter or an ultrasonic generating material emitting a first waveform. In various embodiments of the present disclosure, the operation of forming a second waveform in the first waveform includes using a femtosecond laser to excite a second waveform generating element to emit the second waveform. The ultrasound with complex waveform may apply in ultrasonic imaging and medical application having biological effects. And the ultrasound with complex waveform has the penetrating ability of the low-frequency ultrasound, and the high resolution and the biological medical effects of the high-frequency ultrasound. The biological medical effects include applications such as controlling the ion channel, nanolization the protein precipitate and stem cells renewal.

In various embodiments of the present disclosure, the operation of forming a second waveform in the first waveform includes using a second waveform generating element to adjust the first waveform periodically. Including the operation of using the second waveform generating element to periodically cancel, turn off, or regulate the first waveform. In various embodiments of the present disclosure, the operation of using the second waveform generating element to periodically regulate the first waveform includes doping, casting, blending, or epitaxy growing the second waveform generating element in the first waveform generating element. The first waveform generating element is an ultrasonic generating material, and the second waveform generating element is an oscillating material. The concrete embodiments may refer to the embodiments shown in FIGS. 2-4. In the embodiments, the ultrasonic generating material is a first piezoelectric material, the oscillating material is a phononic crystal, a semiconductor material, or a second piezoelectric material, wherein the frequency of the waveform generated by the second piezoelectric material is higher than that of the first piezoelectric material. The oscillating material may be doped, casted, blended, epitaxy grown, or coupled into the ultrasonic generating material. When the first waveform is generated by connecting with the electricity, the oscillating material may also be operated in the same time to form the second waveform in the first waveform, and generates the ultrasound with complex waveform.

In various embodiments of the present disclosure, the second waveform generating element may be used to periodically turn off the first waveform, and forms the second waveform in the first waveform. The first waveform generating element is an ultrasonic transmitter, and the second waveform generating element is a high frequency switch. The embodiments may refer to FIG. 10.

In various embodiments of the present disclosure, the second waveform generating element emits a pulsed ultrasound periodically to cancel part of the first waveform, and forms the second waveform in the first waveform. In the embodiments, the second waveform generating element is a pulsed ultrasonic transmitter, which may refer to the embodiments in FIG. 12. In various embodiments of the present disclosure, such as embodiments in FIGS. 8, 9 and 11, the second waveform generating element is a chopper, filter, function generator or combinations thereof. The second waveform generating element may cancel part of the first waveform periodically. For example, changing the input signals or segmenting part of the waveforms may also have the effect like the embodiments. The above-mentioned embodiments is not used to limit the present disclosure. Other methods using two ultrasounds with different frequencies to form an ultrasound with complex waveform having the high-frequency waveform in the low-frequency waveform are also protected by the present disclosure.

In various embodiments of the present disclosure, the ultrasonic generating device is provided. The ultrasonic generating device may emit the ultrasound with complex waveform. The ultrasound with complex waveform includes the first waveform and at least one second waveform coupled or in the first waveform. The frequency of the first waveform is in a range from about 15 MHz to about 150 kHz. The frequency of the second waveform is in a range from about 15 MHz to about 250 THz. The ultrasound with complex waveform therefore has penetrating ability of the low-frequency ultrasound which can penetrate into the object, and carrying the second waveform coupled in the first waveform to the object. Because the high-frequency ultrasound with different waveforms and frequencies may have different medical effects, the method of carrying a high-frequency waveform in the low-frequency waveform may be applied in a non-invasive medical treatment. The medical treatment has biological effects such as eliminating tumor chronically in the specific part of the body, enhancing the activity of the proteolytic enzyme, assisting protein refolding correctly. In various embodiments of the present disclosure, the ultrasonic imaging device is provided, which includes the ultrasonic array transmitter and the ultrasonic array receiver. The ultrasonic array transmitter includes a plurality of the ultrasonic generating devices emitting a plurality of the ultrasounds with complex waveforms. The ultrasound with complex waveform is coupled with the high-frequency ultrasound and the low-frequency ultrasound. Therefore, the ultrasounds with complex waveform conserves the penetrating ability of the low-frequency ultrasound which can penetrate into the human body, and has short-wavelength advantage of the high-frequency ultrasound which may enhance the resolution of the ultrasonic imaging device. And the ultrasonic imaging device may apply in an ultrasonic operation using ultrasound to assist orientation and a 3D or 4D real-time imaging.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An ultrasonic generating device for generating an ultrasound with complex waveform comprising:
a first waveform generating element emitting a first waveform; and
a second waveform generating element periodically adjusting the first waveform with at least one frequency higher than that of the first waveform to form a second waveform in the first waveform, and the two waveforms are superposed to form the ultrasound with complex waveform,
wherein a frequency of the second waveform is in a range from about 15 MHz to about 250 THz, and a frequency of the first waveform is in a range from about 15 MHz to about 150 kHz.

2. The ultrasonic generating device of claim 1, wherein the first and the second waveforms are selected from a group consisting of the following waveforms: a square wave, a sinusoidal wave, a triangular wave, a pulse wave and combinations thereof.

3. The ultrasonic generating device of claim 2, wherein the first waveform generating element is an ultrasonic generating material.

4. The ultrasonic generating device of claim 3, wherein the second waveform generating element is an oscillating material, and the oscillating material is distributed into the ultrasonic generating material or coupled to the ultrasonic generating material.

5. The ultrasonic generating device of claim 4, wherein the ultrasonic generating material is a first piezoelectric material.

6. The ultrasonic generating device of claim 5, wherein the first piezoelectric material is selected from a group consisting of quartz, tourmaline, Rochelle salt, tantalate, niobate, titanate and combinations thereof.

7. The ultrasonic generating device of claim 5, wherein the first piezoelectric material is a piezoelectric thin-film material or a polymer piezoelectric film.

8. The ultrasonic generating device of claim 7, wherein the piezoelectric thin-film material is selected from a group consisting of aluminum nitride, zinc oxide, polyvinylidene fluoride (PVDF) and combinations thereof.

9. The ultrasonic generating device of claim 4, wherein the oscillating material is a phononic crystal, a semiconductor material, or a second piezoelectric material, wherein a frequency of a waveform generated by the second piezoelectric material is larger than that of a waveform generated by the first piezoelectric material.

10. The ultrasonic generating device of claim 9, wherein the second piezoelectric material is a piezoelectric thin-film material or a polymer piezoelectric film.

11. The ultrasonic generating device of claim 10, wherein the piezoelectric thin-film material is selected from a group consisting of aluminum nitride, zinc oxide, polyvinylidene fluoride (PVDF) and combinations thereof.

12. The ultrasonic generating device of claim 9, wherein the second piezoelectric material is selected from a group consisting of quartz, tourmaline, Rochelle salt, tantalite, niobate, titanate and combinations thereof.

13. The ultrasonic generating device of claim 9, wherein the semiconductor material is selected from a group consisting of gallium nitride, Indium gallium nitride, silicon carbide and combinations thereof.

14. The ultrasonic generating device of claim 4, further comprising a phase-oppositing ultrasonic transmitter emitting a phase-oppositing ultrasound which is out-of-phase relative to that of the first waveform, and a pathway of the phase-oppositing ultrasound overlaps partially or totally with that of the ultrasound with complex waveform.

15. The ultrasonic generating device of claim 1, wherein the first waveform generating element is a first ultrasonic transmitter.

16. The ultrasonic generating device of claim 15, wherein the second waveform generating element is a periodical waveform regulator, and the periodical waveform regulator connects with the first ultrasonic transmitter.

17. The ultrasonic generating device of claim 16, wherein the periodical waveform regulator is selected from a group consisting of a second ultrasonic transmitter, a filter, a function generator, a high-frequency switch, a chopper and combinations thereof.

18. The ultrasonic generating device of claim 17, wherein the second ultrasonic transmitter is a pulsed ultrasonic transmitter.

19. The ultrasonic generating device of claim 16, further comprising a phase-oppositing ultrasonic transmitter emitting a phase-oppositing ultrasound which is out-of-phase relative to that of the first waveform, and a pathway of the phase-oppositing ultrasound overlaps partially or totally with that of the ultrasound with complex waveform.

20. An ultrasonic imaging device comprising:
an ultrasonic array transmitter comprising a plurality of ultrasonic generating devices, the ultrasonic generating device comprising:
a first waveform generating element emitting a first waveform; and
a second waveform generating element periodically adjusting the first waveform with at least one frequency higher than that of the first waveform to form a second waveform in the first waveform, and the two waveforms are superposed to form an ultrasound with complex waveform,
wherein a frequency of the second waveform is in a range from about 15 MHz to about 250 THz, and a frequency of the first waveform is in a range from about 15 MHz to about 150 kHz; and
an ultrasonic array receiver comprising a plurality of ultrasonic receiving devices to receive the ultrasound with complex waveform reflecting from an area to be imaged.
